(19) United States
(12) Patent Application Publication
(10) Pub. No.: US 2008/0132856 A1
(43) Pub. Date: Jun. 5, 2008

(54) Ostomy Support Garment

(76) Inventors: Bjarne Worsoee, Tikoeb (DK); Michael Hansen, Gilleleje (DK)

Correspondence Address: JACOBSON HOLMAN PLLC, 400 SEVENTH STREET N.W., SUITE 600, WASHINGTON, DC, 20004, US

(21) Appl. No.: 10/565,794

(22) PCT Filed: Feb. 3, 2004
(86) PCT No.: PCT/DK04/00071

§ 371 Date: Jan. 7, 2008

(30) Foreign Application Priority Data
Feb. 3, 2003 (DK) PA 2003 00144

(51) Int. Cl. A61F 5/449

(52) U.S. Cl. 604/343; 604/345

(57) ABSTRACT

An ostomy support set comprising a garment having a first hole for receiving a stoma, said hole having a stabilised edge, and a disc for providing the supporting effect of the material removed for forming the first hole, said disc having a second hole for receiving the stoma wherein the disc has an outer diameter greater than the diameter of the first hole provides a snug fit to the stoma, ensures an easy application and removal of the garment and of a collecting bag combined with a sufficient support next to the stoma.

### BACKGROUND OF THE INVENTION

1. Field of the Invention

The present invention relates to an ostomy support garment having a hole for receiving a stoma and a method for applying an ostomy support garment to a user.

In connection with surgery for a number of diseases in the gastrointestinal tract a consequence is, in many cases that the colon, the ileum or the urethra has been exposed surgically and the patient is left with an abdominal stoma. Such artificial openings or fistulae cannot be controlled at will and are therefore of necessity incontinent and the effluents or waste products of the body, which are conveyed through these organs, are discharged through the artificial orifice or opening and are collected in a collection bag. Said bag is usually adhered to the skin by means of an adhesive wafer or plate having an inlet opening for accommodating the stoma. Such appliances may be two-piece or one-piece appliances. In both types of appliances, a body side member is attached to the wearer's abdomen, and a receiving member or bag is attached to the body side ostomy member for receiving exudates from the stoma. Said receiving member being attached releasably in case of a two-piece appliance.

In many instances patients having had a surgery resulting in the formation of a stoma, an accompanying condition is formation of a peristomal bulge or hernia, which may complicate the bandaging of the stoma and even require further surgery. Even if further surgery is carried out there is a considerable risk of a permanent condition, which cannot be alleviated.

In such cases, the patient will have to rely on an additional hernia support for a mechanical reposition of the bulge or hernia for reducing the risk of constriction or strangulation calling for urgent surgery and for providing a plane surface around the stoma for application of a collecting appliance in order to secure a proper adherence and sealing. Stomal bulge or hernia supports are commonly known and may e.g. be in the form of a belt of e.g. of leather with buckles or in the form of a support garment made from an elastic fabric being able to apply a sufficient pressure around the stoma.

In the case of a colostomy and in case the ostomate is normally irrigating, a minor cap or collecting bag may be used which enables the use of a firm support belt or tight compression briefs for providing a sufficient pressure around the stoma. For ileostomates or urostomates this procedure is not practicable due to the constant rather high output from the ileum or bladder and for urostomates it may be critical to provide a free flow from the stoma in order to prevent a build-up of a backpressure, which may destroy the kidneys.

In such cases, it is highly desirable or mandatory to give access to a larger collecting volume, which means that the collecting bag itself will have to be situated outside the pressure establishing belt or briefs and that a passageway through the same has to be established.

Determination of the site for placing the stoma is normally carried out prior to the operation after observing the patient in different postures, e.g. sitting, standing and bending over, finding the less critical area. WO 00/67683 discloses a device for use in the determination of the optimum position of a stoma-to-be for the patient in question.

As the placing of a stoma is not standardised but depends on the condition and the topography of the abdominal area of the patient, it is not possible to provide a simple selection of standard bulge or hernia supports fitting the majority of patients. The passageways must be tailored according to the actual conditions of the individual patient.

When making a hole in an ostomy support garment it has to be considered that the supporting effect of the missing material has to be provided for in another way and furthermore, it is necessary to stabilise the edge of a hole in order to avoid that it is inadvertently enlarged. At the same time, the effect of the stretching of the support garment and deformation of the shape of the hole when applied has to be taken into consideration as well as the problems associated with providing a sufficiently large hole for allowing an easy passing of an ostomy collection bag during application and removal of the garment and the passing of intestinal contents from the stoma into the bag and at the same time providing a sufficiently snug fit to the stoma to ensure the support next to the stoma.

2. Description of the Related Art

DK Patent Application No. PA 1999 01559 discloses a stomal hernial support compression garment in the form of a pair of compression trousers having a customised hole. The edge of the hole is stabilised by incorporation of a string of nylon sewn with a lockstitch and a zigzag stitch to ensure that the shape of the hole is not changed and furthermore, an enforcement of cotton is sewn using zigzag stitch for stabilising the area around the hole.

U.S. Pat. No. 5,135,520 discloses a variable closure device for an ostomy garment having a pair of criss-cross pocket forming panels configured to lie behind an ostomy device. The criss-cross arrangement of pocket panels are created by finished edges which overlap along their lower ends to define an adjustable, elongated, slanted slot.

It is an object for the present invention to provide a hernia supporting device which provides a hole through which an ostomy bag may be passed, but which is shaped such that the bag is not moved out of the hole by accident. Thus it is an object to provide a hole with an unbroken edge.

Furthermore it is an object of the present invention to provide both a solution wherein the location of the hole in the garment is not critical and a solution offering a snug fit between the ostomy device and the ostomy supporting set.

### SUMMARY OF THE INVENTION

The present invention relates to an ostomy support garment having a hole for receiving a stoma, said hole having a stabilised edge.

Furthermore, the invention relates to a method for applying an ostomy support garment and an ostomy appliance to a user having a stoma and a peristomal bulge or hernia using a two piece ostomy appliance comprising a body side member comprising a hole for receiving the stoma and a receiving bag and an ostomy support garment having a hole for receiving a stoma, said hole having a stabilised edge.

### BRIEF DESCRIPTION OF THE DRAWINGS

The invention is disclosed more in detail with reference to the drawings in which

FIG. 1 shows a sectional view of an embodiment of an ostomy support garment of the invention placed on an ostomate's abdomen together with a two-piece ostomy appliance.

### DETAILED DESCRIPTION OF THE PRESENT INVENTION

The present invention relates to an ostomy support set comprising a garment having a first hole for receiving a stoma, said hole having a stabilised edge, and a disc for providing the supporting effect of the material removed for forming the first hole, said disc having a second hole for receiving the stoma wherein the disc has an outer diameter greater than the diameter of the first hole.

It may be seen as advantage of the present invention that the hole through which an ostomy bag may be passed is provided as a hole with an unbroken edge and thus the bag is not moved out of the hole by accident. Had the hole been defined by a plurality of materials movable in relation to each other such materials could be displaced and thus not provide the snug fit.

Furthermore it may be seen as an advantage of the present invention that the location of the hole in the garment material is not critical as the snug fit is provided by the second hole of the disc. Thus it is easier to mass produce the trousers made of the garment or if the trousers are tailor made the tailor need not to make precise measurements for the determination of the optimal position of the hole.

The support garment set of the invention provides a snug fit to the stoma, ensuring an easy application and removal of the garment and of a collecting bag combined with a sufficient support next to the stoma. Furthermore, the support of the garment set of the invention is independent of stretching of the garment and of the hole.

Thus, the disc having a hole for receiving the stoma may be available in a standard array of sizes, e.g. matching the sizes of the coupling rings of standard ostomy appliances, rendering it easy to choose a disc having a hole of a suitable size. Furthermore, the use of a set comprising a separate disc according to the present invention renders it simple to tailor an ostomy support garment, as it is only necessary to locate the site of the first hole to be made in the support garment. The size of the first hole becomes less critical as the fit to the size of the stoma is provided through the choice of disc, and the hole only needs to be small enough to provide a safe securing of the disc. Thus holes may be made of the maximum size expected to be used, and furthermore, minor changes of the size and shape of a peristomal bulge or hernia is less critical as the combination of the set comprising the disc and the support garment provides for some flexibility with respect to the localisation of the stoma in the hole. Furthermore, a collecting bag is easily pulled through the first hole.

The ostomy support garment of the set of the invention may e.g. be a piece of elastic fabric known per se which may be used by wrapping the piece around the torso of an ostomate and covering the stoma and is preferably in the form of a pair of ostomy support briefs being capable of providing a sufficient support next to the stoma.

The stabilisation of the edge of the hole of the support garment may be made in a manner known per se.

It is normally preferred that the second hole is placed centrally in the disc. For special cases it may be preferred to place the hole of the disc asymmetrically, especially in case of a stoma placed in the abdominal area near the groin.

The diameter of the first hole is preferably greater than the diameter of the second hole. Thus, the rim of the first hole will not interfere with the passing of an ostomy bag during application and removal of the garment or the bag.

The rim of the second hole may be provided with a protruding rim projecting from the plane of the disc, said protruding rim locking the disc and support garment from moving freely, as long as it does not protrude so far as to protrude from the surface of the garment so as to be partly visible which may be embarrassing and may potentially cause problems with trousers or other further clothing.

The disc suitably has an outer diameter of about 110 mm and the second hole suitably a diameter between 50 and about 75 mm depending on the size of the actual coupling system. The hole size should not jeopardize the supporting effect of the disc and at the same time allow a reasonably unproblematic passing a collecting bag during application and removal of the ostomy garment.

When using the ostomy support garment set of the invention together with a two-piece appliance comprising a body side member and a receiving bag which may be attached via a coupling ring it is preferred that the diameter of the second hole is greater than the general outer diameter of the coupling ring of the ostomy body side member in order not to interfere with a safe coupling and also to allow a substitution of the collecting bag without removing the ostomy support garment.

Furthermore, the second hole is preferably smaller than the diameter of an adhesive wafer of the body side member.

The diameter of the second hole may be smaller than the maximum diameter of a coupling system as long as it is large enough not to jeopardize the proper functioning of the coupling system.

The coupling rings may be any system known per se for attaching receiving bags to ostomy body side members and may suitably be matching coupling rings of the type disclosed in WO 94/18919.

Corresponding dimensional considerations apply when using the ostomy support garment set of the invention together with a two-piece appliance comprising a body side member and a receiving bag being attached via matching flanges for adhesive connection e.g. of the type disclosed in U.S. Pat. No. 5,800,415.

When designing ostomy support garments for such cases, care should be taken that the coupling parts be kept essentially plane when the garment is applied in order that the sealing properties of the adhesive coupling are preserved.

The disc of the set of the invention may suitably be made from a material which is not easily soiled if brought into contact with visceral contents and which is easily cleaned, e.g. a polyolefin such as polyethylene, polypropylene, or polybutylene or PVC or a blend or a copolymer thereof. Polyethylene and polypropylene are especially suitable as they are cheap, easy to process into the desired shapes, relatively inert and easy to clean. The disc may be made in a manner known per se, e.g. by injection moulding or pressing using corresponding moulds.

In a second aspect the invention relates to a method for applying an ostomy support garment and an ostomy appliance to a user having a stoma and a peristomal bulge or hernia using a two piece ostomy appliance comprising a body side member comprising a hole for receiving the stoma and a receiving bag and an ostomy support set comprising a garment having a first hole for receiving a stoma, said hole having a stabilised edge, and a disc for providing the supporting effect of the material removed for forming the first hole, said disc having a second hole for receiving the stoma, said disc having an outer diameter greater than the diameter of the first hole which method comprises locating the stoma and placing the body side member on the users abdomen with the stoma projecting through the hole thereof, applying the collecting bag, aligning the hole of the disc with the stoma, pulling the collecting bag through the hole of the disc, placing the disc on the abdomen of the patient with the stoma projecting through the hole and placing and applying the ostomy support garment in such a manner that the stoma projecting through the hole of the disc also projects through the first hole of the ostomy support garment.

In a modification of the second aspect of the invention a one-piece ostomy appliance is used in which case the ostomy appliance is placed on the users abdomen with the stoma projecting into the collecting bag, the hole of the disc is aligned with the stoma, the collecting bag is pulled through the hole of the disc, the disc is placed on the abdomen of the patient with the stoma projecting through the hole and the ostomy support garment is placed and applied in such a manner that the stoma projecting through the hole of the disc also projects through the first hole of the ostomy support garment.

### DESCRIPTION OF THE PREFERRED EMBODIMENTS

The invention is now explained more in detail with reference to the drawings showing preferred embodiments of the invention.

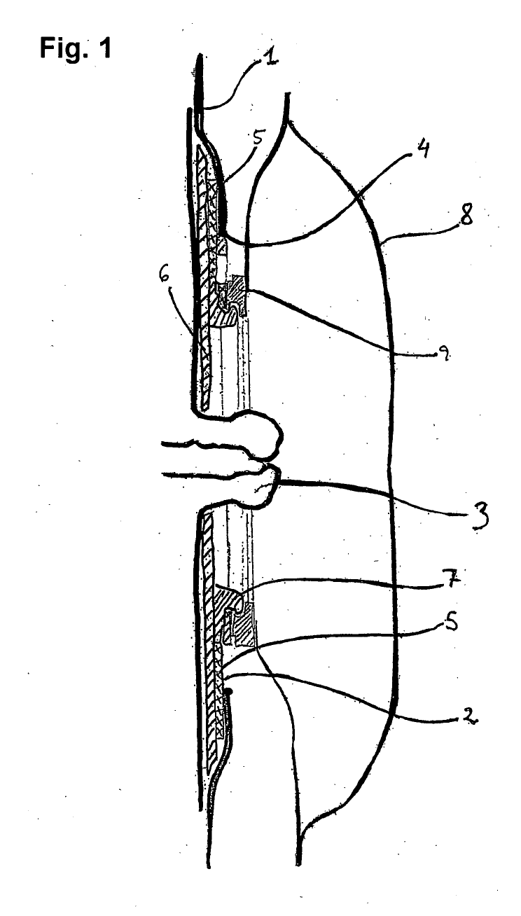

Reference is made to FIG. 1 showing a sectional view of an embodiment of an ostomy support garment set of the invention placed on an ostomate's abdomen together with a two-piece ostomy appliance.

FIG. 1 shows an ostomy support set comprising a garment 1 having a first hole 2 for receiving a stoma 3, said hole having a stabilised edge 4, and a disc 5 having a second hole for receiving the stoma for providing the supporting effect of the material removed for forming the hole of the support garment. Between the support garment and the patients abdomen is placed an ostomy body side member having an adhesive wafer 6 for adhering to the user's skin. The wafer is provided with a coupling ring 7 for releasable attachment of an ostomy collecting bag 8 having a matching coupling ring 9. As can be seen, the diameter of the second hole is greater than the general outer diameter of the coupling ring of the ostomy body side member in order not to interfere with a safe coupling. This allows a substitution of the collecting bag without removing the ostomy support garment.

1: An ostomy support set comprising a garment having a first hole for receiving a stoma, said hole having a stabilised edge, and a disc for providing the supporting effect of a material removed for forming the first hole, said disc having a second hole for receiving the stoma wherein the disc has an outer diameter greater than the diameter of the first hole.

2: An ostomy support garment set as claimed in claim 1 wherein the second hole is placed centrally in the disc.

3: An ostomy support garment set as claimed in claim 1 wherein the diameter of the first hole is greater than the diameter of the second hole.

4: An ostomy support garment set as claimed in claim 1 for use together with a two-piece appliance comprising a body side member and a receiving bag which may be attached via a coupling ring wherein the diameter of the second hole is greater than the diameter of the coupling ring of the ostomy body side member.

5: A method for applying an ostomy support garment and an ostomy appliance to a user having a stoma and a peristomal bulge or hernia using a two piece ostomy appliance comprising a body side member comprising a hole for receiving the stoma and a receiving bag and an ostomy support set comprising a garment having a first hole for receiving a stoma, said hole having a stabilised edge, and a disc for providing the supporting effect of the material removed for forming the first hole, said disc having a second hole for receiving the stoma, said disc having an outer diameter greater than the diameter of the first hole which method comprises locating the stoma and placing the body side member on the users abdomen with the stoma projecting through the hole thereof, applying the collecting bag, aligning the hole of the disc with the stoma, pulling the collecting bag through the hole of the disc, placing the disc on the abdomen of the patient with the stoma projecting through the hole and placing and applying the ostomy support garment in such a manner that the stoma projecting through the hole of the disc also projects through the first hole of the ostomy support garment.

6: An ostomy support garment set as claimed in claim 2 wherein the diameter of the first hole is greater than the diameter of the second hole.

7: An ostomy support garment set as claimed in claim 2 for use together with a two-piece appliance comprising a body side member and a receiving bag which may be attached via a coupling ring wherein the diameter of the second hole is greater than the diameter of the coupling ring of the ostomy body side member.

8: An ostomy support garment set as claimed in claim 3 for use together with a two-piece appliance comprising a body side member and a receiving bag which may be attached via a coupling ring wherein the diameter of the second hole is greater than the diameter of the coupling ring of the ostomy body side member.
